(19) United States
(12) Patent Application Publication
(10) Pub. No.: US 2003/0119846 A1
(43) Pub. Date: Jun. 26, 2003

(54) Compounds with 5-ht activity useful for controlling visual field loss

(76) Inventors: Robert J. Collier Jr, Arlington, TX (US); Mark R. Hellberg, Highland Village, TX (US); Thomas R. Dean, Weatherford, TX (US)

Correspondence Address: ALCON RESEARCH, LTD., R&D COUNSEL, Q-148, 6201 SOUTH FREEWAY, FORT WORTH, TX, 76134-2099, US

(21) Appl. No.: 10/221,056

(22) PCT Filed: Feb. 23, 2001
(86) PCT No.: PCT/US01/05740

(30) Foreign Application Priority Data
Mar. 17, 2000 (US) 60190279

(51) Int. Cl. A61K 31/506; A61K 31/4178; A61K 31/137; A61K 31/557

(52) U.S. Cl. 514/252.15; 514/651; 514/397; 514/573; 514/649

(57) ABSTRACT

Compounds with 5-HT_{1A} agonist activity which are useful for controlling the visual field loss associated with glaucoma are disclosed.

The present invention is directed to compounds with 5-HT_{1A} agonist activity useful for controlling the visual field loss associated with glaucoma.

### BACKGROUND OF THE INVENTION

Glaucoma is a family of diseases, each of which is distinguished by a particular characteristic of that disease form. Primary open angle glaucoma (POAG) is characterized by typical glaucomatous changes to optic nerve head topography, arcurate scotomas in the visual field, an open angle, and is usually associated with elevated intraocular pressure (IOP). Normotension glaucoma (NTG) or low tension glaucoma is very similar to POAG except the IOP for these patients is in the normal range. Other forms of glaucoma include closed angle glaucoma and pigmentary dispersion glaucoma. All these forms of glaucoma are similar in that patients suffer from the continued loss of nerve fiber layer and visual field. Current therapies for the treatment of glaucoma, in particular POAG and NTG, strive to slow the progression of the visual field loss by lowering and controlling intraocular pressure. This is done either by IOP lowering drugs or by argon laser trabeculoplasty (ALT) and/or by glaucoma filtration surgery (GFS). Long-term studies of the effects of lowering IOP (even in NTG patients) have been shown to be effective in slowing the disease progression in some patients. Unfortunately, there are patients who continue to loose visual field despite having their IOP lowered.

MD (mean deviation) and CPSD (corrected pattern standard deviation) are global indices provided by the Humphrey Field Analyzer statistical package. CPSD is a measure of how much the total shape of the patient's visual field deviates from that of the age-matched, normative reference field. If the sensitivity gradient is irregular (as occurs in a scotoma due to glaucoma), a higher CPSD will be recorded. Values for CPSD are positive, and approximate zero in a normal visual field. MD is a measure of the average elevation or depression of the patient's overall field compared to the normal reference field. Values for MD are approximately zero in a normal field and can be either positive or negative. Positive values for MD indicate that the patient's overall field is better than that of the normal age-corrected reference field, while negative values indicate that the patient's overall field is worse than that of the normal age-corrected reference field.

Glaucoma is believed to result in areas of localized loss rather than diffuse loss of sensitivity of the visual field. Since MD is unable to differentiate between a deep localized loss (scotoma due to glaucoma) or diffuise widespread loss (resulting from a small pupil size, uncorrected refractive error, development of cataract, etc.), CPSD is more relevant and useful in detecting and tracking early to moderate glaucomatous visual field loss. Once field loss has reached a significant level of severity (CPSD>10 dB and MD<−25 dB) analysis of the CPSD is no longer useful since with increased severity of loss, the localized nature of the loss is diminished.

The Eye Care Technology Forum has specifically recommended that for studies of glaucoma and ocular hypertension (OHT), analysis procedures be based on localized changes, such as are indicated by the CPSD. See, Johnson, Ophthalmology, Vol. 103, No. 1 (January, 1996).

Drug therapies that both lower IOP and provide additional protection to the retina and optic nerve head have been developed. Compounds such as betaxolol and brimonidine have been shown to be neuroprotective in animal models. Both have been suggested to provide neuroprotection in glaucoma by direct penetration to the back of the eye after topical ocular administration. Betaxolol's neuroprotection properties are believed to arise from its calcium channel blocking activities and its ability to stimulate the expression of key neuroprotective factors such as CNTF, bFGF, and BDNF. Brimonidine is an α₂ agonist and is believed to stimulate the production of bFGF.

Serotonergic 5-HT_{1A} agonists have been reported as being neuroprotective in animal models and many of these agents have been evaluated for the treatment of acute stroke among other indications. This class of compounds has been disclosed for the treatment of glaucoma (lowering and controlling IOP), see e.g., WO 98/18458 (DeSantis, et al) and EP 0771563A2 (Mano, et al.). Osborne, et al. (Ophthalmologica, Vol. 210:308-314, 1996) teach that 8-hydroxydipropylaminotetralin (8-OH-DPAT) (a 5-HT_{1A} agonist) reduces IOP in rabbits. Wang, et al. (Current Eye Research, Vol. 16(8):769-775, August 1997, and IVOS, Vol. 39(4), S488, March, 1998) disclose that 5-methylurapidil, an α_{1A} antagonist and 5-HT_{1A} agonist lowers IOP in the monkey, but due to its α_{1A} receptor activity. Also, 5-HT_{1A} antagonists are disclosed as being useful for the treatment of glaucoma (elevated IOP) (e.g. WO 92/0338, McLees). Furthermore, DeSai, et al. (WO 97/35579) and Macor, et al. (U.S. Pat. No. 5,578,612) disclose the use of 5-HT₁ and 5-HT₁₋ₗᵢₖₑ agonists for the treatment of glaucoma (elevated IOP). These anti-migraine compounds are 5-HT_{1B,D.E.F} agonists, e.g., sumatriptan and naratriptan and related compounds.

### SUMMARY OF THE INVENTION

This invention is directed toward compounds that have potent agonist activity at 5-HT_{1A} receptors. The compounds are useful for controlling the visual field loss associated with glaucoma. The Compounds can be delivered systemically or locally.

### DESCRIPTION OF PREFERRED EMBODIMENTS

Unexpectedly, we have demonstrated that 5-HT_{1A} agonists exhibit potent neuroprotective activity in the eye and as such have utility in controlling the visual field loss associated with glaucoma.

The factors that lead to visual field loss in glaucoma are varied. There are a number of hypothesis that have been put forth over the years to explain glaucoma, however, none of these have been proven to be causative. Visual field loss is a direct consequence of the death (or dysfunction) of the neural retina, in particular retinal ganglion cells. Thus, drug therapies that protect retinal ganglion cells are considered to be useful. Given the fact that glaucoma is a poorly understood disease it is not surprising that there are no well established animal models of the disease. Thus, models that provide insight into mechanism and drug classes that are protective of the neural retina serve as surrogate glaucoma models. The light induced retinopathy model is one of a few such models. This model helps to characterize the ability of a test item to protect the neural retina and, as such, compounds that are active in this model are said to be neuroprotective.

The invention contemplates the use of any pharmaceutically acceptable 5-HT_{1A} agonist, including pharmaceutically acceptable salts, for controlling the visual field loss associated with glaucoma (Compounds). Pharmaceutically acceptable means the Compounds can be safely used for the chronic treatment of glaucoma.

Compounds of the present invention have potent affinity for 5-HT_{1A} receptors with IC₅₀ values that range up to about 500 nM (preferably less than 100 nM). These Compounds are also either full or partial agonists with IC₅₀ values ranging up to about 1 μM (preferably less than 500 nM). Representative 5-HT_{1A} agonists useful according to the present invention include, but are not limited to: tandospirone, urapidil, ziprasidone, repinotan hydrochloride, xaliproden hydrochloride (SR-57746A), buspirone, flesinoxan, EMD-68843, DU-127090, gepirone, alnespirone, PNU-95666, AP-521, flibanserin, MKC-242, lesopitron, sarizotan hydrochloride, Org-13011, Org-12966, E-5842, SUN-N4057, and 8-OH-DPAT₁.

Receptor binding and agonist activity according to this invention can be determined using the following methods.

### METHOD 1

### 5-HT_{1A} Receptor Binding Assay

5-HT_{1A} binding studies were performed with human cloned receptors expressed in Chinese hamster ovary (CHO) cells using (³H)8-OH DPAT as the ligand. Membranes from Chinese hamster ovary cells (CHO) expressing cloned 5-HT_{1A} receptors (manufactured for NEN by Biosignal, Inc., Montreal, Canada) were homogenized in approximately 40 volumes of 50 mM Tris pH 7.4 for 5 sec. Drug dilutions were made using a Beckman Biomek 2000 robot (Beckman Instruments, Fullerton, Calif.). Incubations were conducted with membrane prep, test compounds, and 0.25 nM [³H]8-OH-DPAT (NEN, Boston, Mass.) in the same buffer at 27° C. for 1 h. Assays were terminated by rapid vacuum filtration over Whatman GF/B glass fiber filters pre-soaked in 0.3% polyethyleneimine. Bound radioactivity was measured using liquid scintillation spectrometry. Data were analyzed using non-linear curve fitting programs (Sharif et al., J. Pharmac. Pharmacol. 51:685-694, 1999).

Ligand binding studies can also be run using membrane preparations from calf and rat brain (local source) and human cortex membranes. Specific brain regions were dissected out, homogenized in 10 volumes of 0.32 M sucrose and centrifuged for 10 min at 700×g. The resulting supernatant was centrifuged at 43,500×g for 10 min and the pellet re-suspended in 50 mM Tris-HCl (pH 7.7, 25° C.) using a 10 sec polytron treatment. Aliquots were stored at −140° C. To remove endogenous serotonin, the preps were incubated at 37° C. for 10 min prior to the experiment. Assay incubations were terminated by rapid filtration over Whatman GF/C filters using a Brandel cell harvester. Kᵢ values were calculated using the Cheng-Prusoff equation (De Vry et al., J. Pharm. Exper. Ther. 284:1082-1094, 1998.)

### METHOD 2

### 5-HT_{1A} Functional Assays

The function of Compounds of the present invention can be determined using a variety of methods to assess the functional activity of 5-HT_{1A} agonists. One such assay is performed using hippocampal slices from male Sprague-Dawley rats, measuring the inhibition of forskolin-stimated adenylate cyclase [J. Med. Chem. 42:36 (1999), J. Neurochem. 56:1114 (1991), J. Pharm. Exper. Ther. 284:1082 (1998). Rat hippocampal membranes were homogenized in 25 volumes of 0.3 M sucrose containing 1 mM EGTA, 5 mM EDTA, 5 mM dithiothreitol, and 20 mM Tris-HCl, pH 7.4 at 25° C. The homogenate was centrifuged for 10 m in at 1,000×g. The supernatant subsequently was centrifuged at 39,000×g for 10 min. The resulting pellet was re-suspended in homogenization buffer at a protein concentration of approximately 1 mg/ml and aliquots were stored at −140° C. Prior to use, the membranes were rehomogenized in a Potter-Elvehjem homogenizer. Fifty μl of the membrane suspension (50 μg protein) were added to an incubation buffer containing 100 mM NaCl, 2 mM magnesium acetate, 0.2 mM ATP, 1 mM cAMP, 0.01 mM GTP, 0.01 mM forskolin, 80 mM Tris-HCl, 5 mM creatine phosphate, 0.8 U/μl creatine phosphokinase, 0.1 mM IBMX, 1-2 μCi α-[³²P]ATP. Incubations with test compounds (10 min at 30° C.) were initiated by the addition of the membrane solution to the incubation mixture (prewarmed 5 min at 30° C.). [³²P]cAMP was measured according to the method of Salomon (Adv. Cyclic Nucleotide Res. 10:35-55, 1979). Protein was measure using the Bradford (Anal. Biochem 72:248-254, 1976) assay.

Functional activity can also be determined in recombinant human receptors according to the method of Schoeffter et al., (Neuropharm. 36:429-437, 1997). HeLa cells transfected with recombinant human 5-HT_{1A} receptors were grown to confluence in 24-well plates. The cells were rinsed with 1 ml of Hepes-buffered saline (in MM) NaCl 130, KCl 5.4, CaCl₂, 1.8, MgSO₄ 0.8, NaH₂PO₄ 0.9, glucose 25, Hepes 20, pH 7.4, and phenol red 5 mg/l. The cells were labelled with 6 μCi/ml of [²⁻³H]adenine (23 Ci/mmol, Amersham, Rahn AG, Zurich, Switzerland) in 0.5 ml of saline at 37 ° C. for 2 hr. The plates were subsequently rinsed twice with 1 ml of buffered saline containing 1 mM isobutylmethylxanthine. The cells were incubated for 15 min in 1 ml of this solution (37° C.) in the presence or absence of 10 μM forskolin and the test compound. The buffer was then removed and 1 ml of 5% trichloroacetic acid (TCA) containing 0.1 mM cAMP and 0.1 mM ATP was added to extract the samples. After 30 min at 4° C., the TCA extracts were subjected to chromatographic separation on Dowex AG 50W-X4 and alumina columns (Salomon, Methods in Enzymology 195: 22-28, 1991). Cyclic AMP production was calculated as the ratio [³H]cAMP/([³H]cAMP+[³H]ATP).

The above procedures described in Methods 1 and 2 were used to generate the following data.

TABLE 1
5-HT_{1A} Receptor Binding and Functional Assay Data.

| | Receptor Binding | cAMP Inhibition |
|---|---|---|
| Compound | (IC₅₀ nM, SEM) | (EC₅₀) |
| (R,S) 8-OH-DPAT | 1.5 nM | 4.7 nM |
| (R) 8-OH-DPAT | 0.5 uM | 2.6 nM |
| SR-57746A | 2.5 nM | 3.7 nM |

### METHOD 3

### Neuroprotective effects in the acute blue-light damage model in the rat

Male Sprague Dawley rats were randomly assigned to vehicle treatment (N=15) or drug treatment (0.5 mg/kg [N=5] or 1 mg/kg [N=15]) experimental groups. Xaliproden hydrochloride or vehicle was administered by intraperitonal (IP) injection at 48, 24, and 0 hours prior to light exposure. Photo-oxidative injury to the retina was induced in dark-adapted rats (24 hours) by a 6-hour blue-light exposure (220 fc). Control rats (N=11) were housed in their home cage under normal cyclic light exposure. Rats were single housed in clear polycarbonate cages during this light exposure.

The electroretinogram (ERG) was recorded after a five day recovery period from dark-adapted anesthetized rats (Ketamine-HCl, 75 mg/Kg; Xylazine, 6 mg/Kg). The eye's electrical response to a flash of light was elicited by viewing a ganzfeld. ERGs to a series of light flashes increasing in intensity were digitized to analyze temporal characteristics of the waveform and determine the response voltage-log intensity (VlogI) relationship. Changes in the ERG a-wave are associated with photoreceptor and retinal pigment epithelium damage while damage to the inner retina is reflected in changes in the ERG b-wave.

Blue-light exposure for 6 hours resulted in a significant diminution of the ERG response amplitude (ANOVA, p<0.001; Bonferroni t-test, p<0.05) compared to normals when measured after a 5-day recovery period (Table 2). Blue-light exposure resulted in a 50% reduction in the maximum a- and b-wave amplitudes in vehicle dosed rats compared to controls. In addition, threshold responses were lower and evoked at brighter flash intensities.

Significant protection of retinal function was measured in light-exposed rats dosed with xaliproden hydrochloride (0.5 and 1.0 mg/kg). Maximum a- and b-wave response amplitudes were 82% of controls in rats dosed with SR-57746A (0.5 mg/kg) and 70% of normal in rats dosed with 1 mg/kg.

TABLE 2

| | |
|---|---|
| A-wave | B-wave |
| Amplitude | Amplitude |

| | | Mean | | Mean | |
|---|---|---|---|---|---|
| Treatment | N | (μV) | SEM | (μV) | SEM |
| Control | 11 | 675.8 | 51.9 | 1554.7 | 87.5 |
| Vehicle | 19 | 313.6 | 39.3 | 759.7 | 110.3 |
| Xaliproden | | | | | |
| Hydrochloride | | | | | |
| 0.5 mg/kg | 5 | 550.5 | 39.8 | 1361.1 | 94.2 |
| 1.0 mg/kg | 15 | 460.8 | 39.6 | 1169.4 | 131.1 |

### METHOD 4

### Pharmacokinetic studies in rabbits

New Zealand Albino or Dutch-belted rabbits (3 to 5 per arm) can be dosed topically with a solution formulation of Compound (1%) in the right eye and with vehicle in the left eye twice a day for a period of one week. At the end of the dosing period the ocular fluids and tissues are collected and analyzed for the presence of the drug via HPLC analysis. The difference between the dosed eye and the contralateral vehicle dosed eye is a measure of the ability of the test item to penetrate directly to the retina/optic nerve head via topical ocular drug delivery. The drug concentrations in the vehicle dosed eye represent delivery from systemic circulation.

In general the 5-HT_{1A} agonists of this invention are administered orally with daily dosage of these compounds ranging between about 0.001 and about 500 milligrams. The preferred total daily dose ranges between about 1 and about 100 milligrams. Non-oral administration, such as, intravitreal, topical ocular, transdermal patch, subdermal, parenteral, intraocular, subconjunctival, or retrobulbar or subtenon's injection, trans scleral (including iontophoresis), or slow release biodegradable polymers or liposomes may require an adjustment of the total daily dose necessary to provide a therapeutically effective amount of the Compound. The 5-HT_{1A} agonists can also be delivered in ocular irrigating solutions. Concentrations should range from about 0.001 μM to about 100 μM, preferably about 0.01 μM to about 5 μM.

The 5-HT_{1A} agonists can be incorporated into various types of ophthalmic formulations for delivery to the eye (e.g., topically, intracamerally, or via an implant). They may be combined with ophthalmologically acceptable preservatives, surfactants, viscosity enhancers, gelling agents, penetration enhancers, buffers, sodium chloride, and water to form aqueous, sterile ophthalmic suspensions or solutions or preformed gels or gels formed in situ. Ophthalmic solution formulations may be prepared by dissolving the compound in a physiologically acceptable isotonic aqueous buffer. Further, the ophthalmic solution may include an ophthalmologically acceptable surfactant to assist in dissolving the compound. The ophthalmic solutions may contain a viscosity enhancer, such as, hydroxymethylcellulose, hydroxyethylcellulose, hydroxypropylmethylcellulose, methylcellulose, polyvinyl-pyrrolidone, or the like, to improve the retention of the formulation in the conjunctival sac. In order to prepare sterile ophthalmic ointment formulations, the active ingredient is combined with a preservative in an appropriate vehicle, such as, mineral oil, liquid lanolin, or white petrolatum. Sterile ophthalmic gel formulations may be prepared by suspending the active ingredient in a hydrophilic base prepared from the combination of, for example, carbopol-940, or the like, according to the published formulations for analogous ophthalmic preparations; preservatives and tonicity agents can be incorporated.

If dosed topically, the 5-HT_{1A} agonists are preferably formulated as topical ophthalmic suspensions or solutions, with a pH of about 4 to 8. The 5-HT_{1A} agonists will normally be contained in these formulations in an amount 0.001% to 5% by weight, but preferably in an amount of 0.01% to 2% by weight. Thus, for topical presentation, 1 to 2 drops of these formulations would be delivered to the surface of the eye 1 to 4 times per day according to the discretion of a skilled clinician.

The Compounds can also be used in combination with other agents for treating glaucoma, such as, but not limited to, β-blockers (e.g., timolol, betaxolol, levobetaxolol, carteolol, levobunolol, propranolol), carbonic anhydrase inhibitors (e.g., brinzolamide and dorzolamide), α₁ antagonists (e.g. nipradolol), α2 agonists (e.g., iopidine and brimonidine), miotics (e.g., pilocarpine and epinephrine), prostaglandin analogues (e.g., latanoprost, travaprost, unoprostone, bimatoprost, and compounds set forth in U.S. Pat. Nos. 5,889,052; 5,296,504; 5,422,368; 5,688,819; and 5,151,444, “hypotensive lipids” (e.g., compounds set forth in U.S. Pat. No. 5,352,708), and neuroprotectants (e.g., compounds from U.S. Pat. No. 4,690,931, particularly eliprodil and R-eliprodil, as set forth in a pending application U.S. Ser. No. 06/203350, and appropriate compounds from WO94/13275, such as, memantine.

The following topical ophthalmic formulations are useful according to the present invention administered 1-4 times per day according to the discretion of a skilled clinician.

### EXAMPLE 1

| Ingredients | Amount (wt %) |
|---|---|
| Buspirone | 0.01-2% |
| Hydroxypropyl methylcellulose | 0.5% |
| Dibasic sodium phosphate (anhydrous) | 0.2% |
| Sodium chloride | 0.5% |
| Disodium EDTA (Edetate disodium) | 0.01% |
| Polysorbate 80 | 0.05% |
| Benzalkonium chloride | 0.01% |
| Sodium hydroxide/Hydrochloric acid | For adjusting pH to 7.3-7.4 |
| Purified water | q.s. to 100% |

### EXAMPLE 2

| Ingredients | Amount (wt %) |
|---|---|
| Buspirone | 0.01-2% |
| Methyl cellulose | 4.0% |
| Dibasic sodium phosphate (anhydrous) | 0.2% |
| Sodium chloride | 0.5% |
| Disodium EDTA (Edetate disodium) | 0.01% |
| Polysorbate 80 | 0.05% |
| Benzalkonium chloride | 0.01% |
| Sodium hydroxide/Hydrochloric acid | For adjusting pH to 7.3-7.4 |
| Purified water | q.s. to 100% |

### EXAMPLE 3

| Ingredients | Amount (wt %) |
|---|---|
| Compound | 0.01-2% |
| Guar gum | 0.4-6.0% |
| Dibasic sodium phosphate (anhydrous) | 0.2% |
| Sodium chloride | 0.5% |
| Disodium EDTA (Edetate disodium) | 0.01% |
| Polysorbate 80 | 0.05% |
| Benzalkonium chloride | 0.01% |
| Sodium hydroxide/Hydrochloric acid | For adjusting pH to 7.3-7.4 |
| Purified water | q.s. to 100% |

### EXAMPLE 4

| Ingredients | Amount (wt %) |
|---|---|
| Xaliproden hydrochloride | 0.01-2% |
| White petrolatum and mineral oil and lanolin | Ointment consistency |
| Dibasic sodium phosphate (anhydrous) | 0.2% |
| Sodium chloride | 0.5% |
| Disodium EDTA (Edetate disodium) | 0.01% |
| Polysorbate 80 | 0.05% |
| Benzalkonium chloride | 0.01% |
| Sodium hydroxide/Hydrochloric acid | For adjusting pH to 7.3-7.4 |

### EXAMPLE 5

10 Mm IV Solution w/v %

| | |
|---|---|
| Buspirone | 0.384% |
| L-Tartaric acid | 2.31% |
| Sodium hydroxide | pH 3.8 |
| Hydrochloric acid | pH 3.8 |
| Purified water | q.s. 100% |

### EXAMPLE 6

5 mg Capsules

| Ingredient | mg/capsule |
|---|---|
| Buspirone Hydrochloride | 5 |
| Lactose, anhydrous | 55.7 |
| Starch, Sodium carboxy-methyl | 8 |
| Cellulose, microcrystalline | 30 |
| Colloidal silicon dioxide | .5 |
| Magnesium sterate | .8 |

we claim:

1. A method for controlling the visual field loss associated with glaucoma which comprises administering a pharmaceutically effective amount of a compound with 5-HT_{1A} agonist activity.

2. The method of claim 2 wherein the compound is selected from the group consisting of: tandospirone, urapidil, ziprasidone, repinotan hydrochloride, xaliproden hydrochloride (SR-57746A), buspirone, flesinoxan, EMD-68843, DU-127090, gepirone, alnespirone, PNU-95666, AP-521, flibanserin, MKC-242, lesopitron, sarizotan hydrochloride, Org-13011, Org- 12966, E-5842, SUN-N4057, and 8-OH-DPAT₁.

3. The method of claim 2 wherein the compound is buspirone.

4. The use of a compound with 5-HT_{1A} agonist activity for the manufacture of a medicament for controlling the visual field loss associated with glaucoma.

5. The use of claim 4 wherein the compound is selected from the group consisting of: tandospirone, urapidil, ziprasidone, repinotan hydrochloride, xaliproden hydrochloride (SR-57746A), buspirone, flesinoxan, EMD-68843, DU-127090, gepirone, alnespirone, PNU-95666, AP-521, flibanserin, MKC-242, lesopitron, sarizotan hydrochloride, Org-13011, Org-12966, E-5842, SUN-N4057, and 8-OH-DPAT₁.

6. The use of claim 5 wherein the compound is buspirone.

7. The method of claim 1, 2, or 3 wherein the compounds are used in combination with an additional agent for treating glaucoma.

8. The method of claim 7 wherein the additional agent is selected from the group consisting of: β-blockers (e.g., timolol, betaxolol, levobetaxolol, carteolol, levobunolol, propranolol), carbonic anhydrase inhibitors (e.g., brinzolamide and dorzolamide), α₁ antagonists (erg. nipradolol), α₂ agonists (e.g., iopidine and brimonidine), miotics (e.g., pilocarpine and epinephrine), prostaglandin analogues (e.g., latanoprost, travaprost, unoprostone, bimatoprost, and compounds set forth in U.S. Pat. Nos. 5,889,052; 5,296,504; 5,422,368; 5,688,819; and 5,151,444, “hypotensive lipids” (e.g., compounds set forth in U.S. Pat. No. 5,352,708), and neuroprotectants (e.g., compounds from U.S. Pat. No. 4,690,931, particularly eliprodil and R-eliprodil, as set forth in a pending application U.S. Ser. No. 06/203350, and appropriate compounds from WO94/13275, such as, memantine.

9. The use of claims 4, 5, or 6 wherein the compounds are used in combination with an additional agent for treating glaucoma.

10. The use of claim 9 wherein the additional agent is selected from the group consisting of: β-blockers (e.g., timolol, betaxolol, levobetaxolol, carteolol, levobunolol, propranolol), carbonic anhydrase inhibitors (e.g., brinzolamide and dorzolamide), α₁ antagonists (e.g. nipradolol), α₂ agonists (e.g., iopidine and brimonidine), miotics (e.g., pilocarpine and epinephrine), prostaglandin analogues (e.g., latanoprost, travaprost, unoprostone, bimatoprost, and compounds set forth in U.S. Pat. Nos. 5,889,052; 5,296,504; 5,422,368; 5,688,819; and 5,151,444, “hypotensive lipids” (e.g., compounds set forth in U.S. Pat. No. 5,352,708), and neuroprotectants (e.g., compounds from U.S. Pat. No. 4,690,931, particularly eliprodil and R-eliprodil, as set forth in a pending application U.S. Ser. No. 06/203350, and appropriate compounds from WO94/13275, such as, memantine

11. The use of a compound with 5-HT_{1A} agonist activity for the manufacture of a medicament for providing neuroprotection to the neural retina.

12. The use of claim 11 wherein the neuroprotection is provided in order to control the visual field loss associated with glaucoma.

13. The use of claim 11 or 12 wherein the neural retina comprises retinal ganglion cells.
